(19) United States
(12) Patent Application Publication
(10) Pub. No.: US 2003/0023095 A1
(43) Pub. Date: Jan. 30, 2003

(54) Method for the production of nitroethionate

(76) Inventor: Frank-Hardi Wartenberg, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/203,889

(22) PCT Filed: Feb. 14, 2001
(86) PCT No.: PCT/EP01/01635

(30) Foreign Application Priority Data
Feb. 15, 2000 (DE) 10006545.7

(51) Int. Cl. C07D 275/06

(52) U.S. Cl. 548/207

(57) ABSTRACT

The present invention relates to a process by means of which nitroethionate can be prepared safely by reaction of 4-nitro-N-methylaniline with carbyl sulphate. The process is characterized in that the solvent selected is a nitrile-substituted organic hydrocarbon, an ether of polyhydric alcohols or a cyclic ether. The risk of local overconcentrations, local overheating or explosions is avoided by a homogeneous reaction procedure.

The present invention relates to a novel process for preparing nitroethionate in which acetonitrile is used as solvent.

Nitroethionate is an intermediate in the production of dyes. It is prepared by reacting 4-nitro-N-methylaniline with carbyl sulphate (1,3-dioxa-2,4-dithiane 2,4-bis-dioxide). This is shown in the following equation.

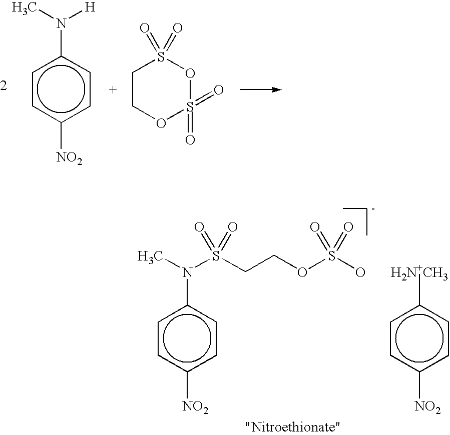

In practice, the reaction is extremely difficult to carry out because of the high reactivity of the carbyl sulphate combined with its sparing solubility or even insolubility in most organic solvents. For example, carbyl sulphate is insoluble in aprotic organic solvents. Neither is it possible to use protic solvents. Although carbyl sulphate dissolves satisfactorily in these, the dissolution is accompanied by simultaneous decomposition which interferes or even completely prevents the reaction.

4-nitro-N-methylaniline, too, displays a problematical dissolution behaviour insofar as it is likewise insoluble in many customary organic solvents, which introduces additional difficulties in selecting a solvent suitable for the reaction of these two reactants.

For the reasons mentioned, attempts to find a solvent for carrying out the reaction shown in the above equation in which both reactants dissolve without decomposition and which makes it possible to carry out the reaction in a homogeneous phase have not been successful up to now.

In the industrial process employed up to now for the synthesis of nitroethionate, recourse is made to nitrobenzene as reaction medium. A solution of 4-nitro-N-methylaniline, in which a high proportion of solid is still present because of the poor solubility, is initially charged. The carbyl sulphate, which is virtually insoluble in nitrobenzene, is subsequently added in solid form a little at a time, with the reaction mixture having to be stirred intensively to avoid possible overconcentrations due to the inhomogeneous reaction procedure. In this reaction procedure, the carbyl sulphate reacts moderately quickly and the participating reactants and the reaction mixture have a satisfactory stability under the reaction conditions selected (temperature of the reaction mixture: about 40-50° C.) as long as sufficient mixing of the reactants is ensured.

Problems can occur in the heterogeneous reaction procedure when sufficient mixing of the reactants is not ensured. This can, for instance, result in lump formation, i.e. in local overconcentrations. If this is the case, spontaneous decomposition or explosion of the substances can occur because of local overheating. This is particularly dangerous at the reactor wall which is generally heated from the outside and at which high local temperatures are found. For example, an explosion of a vessel occurred in the nitroethionate synthesis at Hoechst AG in 1973 and caused injury to people.

However, the process for preparing sodium ethionate has up to now continued to be carried out in the above-described manner with appropriate safety precautions, since no conditions which allow a simple, homogeneous reaction procedure and do not lead to decomposition of the starting materials used or even the products obtained has been found up to now.

It is therefore an object of the present invention to carry out the preparation of nitroethionate from 4-nitro-N-methylaniline and carbyl sulphate in such a way that the occurrence of local overheating or even explosions is avoided and a simple, effective procedure giving high yields is ensured at the same time.

This object is achieved by a process for preparing nitroethionate by reacting 4-nitro-N-methylaniline with carbyl sulphate, characterized in that the reaction is carried out in a nitrile-substituted organic hydrocarbon, an ethyl of polyhydric alcohols or a cyclic ether as solvent.

It has surprisingly been found that the use of the abovementioned solvents ensures a simple, homogeneous and safe reaction procedure which prevents a risk of explosion in the nitroethionate synthesis.

Suitable solvents for the synthesis according to the invention are nitrile-substituted organic hydrocarbons, for example acetonitrile, propionitrile or benzonitrile. Another class of solvents which can be used consists of ethers of polyhydric alcohols. Examples are 1,2-dimethoxyethane(ethylene glycol dimethyl ether), diethylene glycol dimethyl ether and triethylene glycol dimethyl ether. Cyclic ethers such as dioxane or tetrahydrofuran are also suitable.

Preference is given to using acetonitrile or 1,2-dimethoxyethane. The most preferred solvent is acetonitrile.

It has been found that both 4-nitro-N-methyl-aniline and carbyl sulphate dissolve satisfactorily in the solvents mentioned even at room temperature. The dissolution behaviour can be improved still further by slight heating of the solvent, so that a completely homogeneous reaction procedure can be achieved without use of excessive solvent. The temperatures necessary to achieve complete dissolution of the reactants are about 35-55° C., namely in a region in which the thermal stability of both the reactants and the product is ensured.

The homogeneous reaction procedure eliminates the risk of local overheating and overconcentrations which could lead to explosions. A further advantage of the use of the solvents mentioned is that they generally have boiling points which are significantly below the temperature at which exothermic decomposition reactions of the reactants commence. This temperature is about 180° C. while, for example, the boiling point of acetonitrile is 82° C., i.e. significantly below this temperature. Should the selected reaction temperature, generally in the range 40-75° C., rise unexpectedly, the heat of reaction can therefore be removed by vaporization of the solvent. This is not the case when nitrobenzene is used. The latter solvent boils at 210° C., i.e. significantly above the critical temperature of 180° C. which makes spontaneous decomposition reactions possible, thus obviously increasing the risk of decomposition.

To carry out the reaction according to the invention, 4-nitro-N-methylaniline is firstly added to the solvent chosen, preferably acetonitrile, and the solution is heated to from about 35 to 55° C., preferably from 40 to 50° C. The amounts are selected so that the solution contains from 5 to 20% by weight, preferably from 10 to 15% by weight, of starting material. In these concentration ranges, complete dissolution of the starting material can be achieved by slight heating. A solution of carbyl sulphate in the solvent, preferably acetonitrile, is subsequently prepared using amounts such that the solution contains from 20 to 50% by weight, preferably from 20 to 30% by weight, of carbyl sulphate. The solution can likewise be heated to temperatures of from 35 to 55° C., but is preferably kept at room temperature.

The heated, 4-nitro-N-methylaniline-containing solution is placed in the reaction vessel and the carbyl sulphate solution is metered in, generally over a period of from 10 to 30 minutes. The temperature of the reaction solution is 40-75° C., preferably 50° C. After the addition is complete, the solution is then advantageously heated to the boiling point and generally maintained at this temperature for from 30 to 90 minutes, after which the reaction is complete.

After the reaction has ended, the solution is then worked up in an appropriate manner, generally by addition of water and/or Na₂CO₃ solution or K₂CO₃ solution and extraction of the organic phase with a suitable solvent, for instance an ether such as methyl tert-butyl ether. The reaction is preferably carried out under an inert gas atmosphere, for example a nitrogen or argon atmosphere.

The present invention is illustrated by the following examples. The amounts indicated in these examples can be increased without problems by a factor of from 100 to 200 in the process of the invention.

### COMPARATIVE EXAMPLE 1

Under a nitrogen atmosphere, 4-nitro-N-methyl-aniline (760 g) is suspended in 3000 ml of nitro-benzene. This suspension is heated to an internal temperature of 45° C. 470 g of carbyl sulphate are subsequently added a little at a time over a period of one hour while stirring vigorously. The internal temperature increases to 47° C. during the addition. The formation of a yellowish brown solution is observed. After the addition is complete, the reaction mixture is stirred for another two hours at an internal temperature of 70-75° C. to complete the reaction. The reaction mixture is subsequently stirred into ice water, forming a fine precipitate. 155 g of sodium carbonate are sprinkled in and the pH is brought to 5.5-6.0 during a further one hour of stirring. This results in the precipitate going back into solution. The mixture is heated to an internal temperature of 60° C., stirred for 30 minutes, cooled back to room temperature and allowed to settle (pH 6.0). The lower nitrobenzene phase is drained off and stood in a refrigerated room, resulting in excess 4-nitro-N-methylaniline crystallizing out. This is filtered off with suction and washed with ethanol. The upper aqueous phase is washed twice with 2 1 each time of methyl tert-butyl ether and admixed with 70 g of activated carbon and 300 g of kieselguhr. The mixture is heated at 60-65° C. for 30 minutes and filtered off with suction through kieselguhr. The filtrate is admixed with 0.7 kg of KCl and cooled to room temperature while stirring well. This results in precipitation of a major part of the product. The remainder is salted out by addition of 1.4 kg of NaCl and subsequent stirring overnight at 10-15° C. The solid is filtered off with suction and dried at 50-55° C. under reduced pressure.

Yield: 656 g=70% of theory

### EXAMPLE 1

A solution of 60.2 g of 4-nitro-N-methylaniline in 500 ml of acetonitrile is prepared under a nitrogen atmosphere. To completely dissolve the substrate, this solution is heated to 50° C. A solution of 38.2 g of carbyl sulphate in 200 ml of acetonitrile is subsequently metered in at an internal temperature of 50-55° C. over a period of 15 minutes. After the addition is complete, the solution is heated to boiling and held at this temperature for about 60 minutes to complete the reaction. The reaction solution is subsequently allowed to cool slowly. The reaction mixture is then worked up by pouring it into a mixture of 1400 ml of water and 1000 ml of methyl tert-butyl ether and adjusting the pH of the system to 5.5-6.0 by addition of sodium carbonate. After the organic and aqueous phases have separated, the organic phase is separated off and washed with water. The combined aqueous phases are stirred with 7.9 g of activated carbon and 30 g of kieselguhr for 60 minutes at about 60° C., filtered, the hot filtrate is admixed with 65 g of potassium chloride and allowed to cool. This results in precipitation of the product in the form of crystals; for complete precipitation, it may be necessary to add further potassium chloride or sodium chloride.

This gives 45.5 g of crude nitroethionate which is recrystallized from about 150 ml of boiling water. The crystals obtained are washed with methyl tert-butyl ether and dried, giving 38.3 g of product.

1. Process for preparing nitroethionate by reaction of 4-nitro-N-methylaniline and carbyl sulphate, characterized in that the reaction is carried out in a nitrile-substituted organic hydrocarbon, an ether of polyhydric alcohols or a cyclic ether.

2. Process according to claim 1, characterized in that the solvent used is acetonitrile, propionitrile, benzonitrile, 1,2-dimethoxyethane, diethylene glycol dimethyl ether, triethylene glycol dimethyl ether, dioxane or tetrahydrofuran.

3. Process according to claim 1 or 2, characterized in that the solvent used is acetonitrile or 1,2-dimethoxyethane, preferably acetonitrile.

4. Process according to any of claims 1 to 3, characterized in that each of the two reactants is dissolved in the appropriate solvent and these solutions are then combined and reacted.

5. Process according to claim 4, characterized in that the solution of 4-nitro-N-methylaniline contains from 5 to 20% by weight, preferably from 10 to 15% by weight, of substrate.

6. Process according to claim 4 or 5, characterized in that the solution of carbyl sulphate in acetonitrile contains from 20 to 50% by weight, preferably from 20 to 30% by weight, of substrate.

7. Process according to any of claims 4 to 6, characterized in that at least one of the solutions of the reactants is heated to a temperature of from 35 to 55° C. before the solutions are combined.

8. Process according to any of claims 1 to 7, characterized in that the reaction is carried out at a temperature of from 40 to 75° C., preferably 50° C.

9. Process according to any of claims 1 to 8, characterized in that the reaction is carried out over a period of from 10 to 30 minutes.

10. Process according to any of claims 1 to 9, characterized in that it is carried out under an inert gas atmosphere, preferably a nitrogen or argon atmosphere.
